(19) United States
(12) Patent Application Publication
(10) Pub. No.: US 2008/0286219 A1
(43) Pub. Date: Nov. 20, 2008

(54) Hydroquinone derivative skin brightening compounds

(76) Inventors: Michael Fitzpatrick Wempe, Kingsport, TN (US); Jeffrey M. Clauson, Johnson City, TN (US)

Correspondence Address: BRETT L. NELSON;EASTMAN CHEMICAL COMPANY, 100 NORTH EASTMAN ROAD, KINGSPORT, TN, 37660-5075, US

(21) Appl. No.: 11/804,120

(22) Filed: May 17, 2007

(51) Int. Cl. A61K 8/34; A61K 8/49; A61Q 19/02; C07C 43/178; C07C 59/52; C07C 69/66; C07D 207/16; C07D 277/36

(52) U.S. Cl. 424/62; 548/187; 548/540; 560/17; 562/431; 568/46; 568/47

(57) ABSTRACT

Skin brightening compositions based on hydroquinone derivatives and methods of making and using the composition. The hydroquinone derivatives are 2-thio-hydroquinone derivatives.

### FIELD OF THE INVENTION

The present invention relates to skin brightening compositions based on hydroquinone derivatives. Another aspect of the present invention relates to the methods of making the hydroquinone derivatives and the skin brightening composition as well as a method of using the compositions.

### BACKGROUND OF THE INVENTION

Since the 1950's, hydroquinone (HQ) has been used as an over-the-counter (OTC) active for brightening human skin. From a clinical point-of-view, a HQ cream of ≦2% has been considered ‘safe for skin de-pigmentation’ (A. P. DeCaprio (1999) The Toxicology of Hydroquinone—Relevance to Occupational and Environmental. Exposure. Critical Reviews in Toxicology 29(3):283-330 and M. I. Rendon, J. I. Gaviria (2005) Review of Skin-Lightening Agents. Dermatol. Surg. 31: 886-889). However, the Food and Drug Administration (FDA) has recently given notice that it has proposed to ban hydroquinone-containing products, both OTC and prescription (Cosmetic Surgery Times, Nov. 14^{th}2006). The only currently known exception would be Tri-Luma Cream®, the only commercially available HQ-containing product with an approved New Drug Application (NDA). Hence, a HQ ban would create a ripple in the skin-brightening market and require novel replacements.

The present invention addresses the need for new skin-brightening agents by constructing novel HQ related compounds; compounds that possess potent skin-brightening activity. The present inventors have synthetically prepared a variety of novel compounds that contain the hydroquinone core. These compounds are designed with the aim of having an improved clinical safety profile and/or to be more efficacious (i.e. use smaller quantities of material in vivo) than HQ; e.g. by inhibiting tyrosinase and/or to cause reduction in the production of melanin.

### SUMMARY OF THE INVENTION

A first embodiment of the present invention concerns a hydroquinone derivative represented by the following formula:

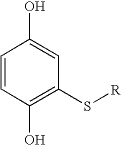

wherein R is a moiety selected from the group consisting of an alkyl, an alkenyl, a dienyl, a trienyl, and a tetraenyl containing up to about 20 carbon atoms, or R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

Another embodiment concerns a composition for brightening skin, comprising a hydroquinone derivative represented by the following formula:

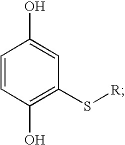

and a cosmetically acceptable carrier, wherein R is a moiety selected from the group consisting of an alkyl, an alkenyl, a dienyl, a trienyl, and a tetraenyl containing up to about 20 carbon atoms, or R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

Yet another embodiment concerns a method of brightening skin, comprising applying the composition according to the present invention to skin.

Another embodiment concerns a method of making a hydroquinone derivative comprising reacting benzoquinone with a molecule having the structure SH—R in the presence of an organic solvent or a mixed organic solvent, wherein R is a moiety selected from the group consisting of an alkyl, an alkenyl, a dienyl, a trienyl, and a tetraenyl containing up to about 20 carbon atoms, or R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

### DETAILED DESCRIPTION

The present invention relates to skin brightening compositions based on hydroquinone derivatives. The hydroquinone derivatives according to the present invention are 2-thio-hydroquinone derivatives represented by the following formula:

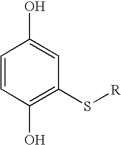

wherein R is selected from alkyl, alkenyl, dienyl, trienyl, and tetraenyl groups which may be straight- or branched-chain aliphatic hydrocarbon moieties containing up to about 20 carbon atoms and may be substituted, for example, with one to three groups selected from C₁-C₆-alkoxy, cyano, C₂-C₆-alkoxycarbonyl, C₂-C₆-alkanoyloxy, hydroxy, aryl, heteroaryl, thiol, thioether, dithiolane, and halogen.

The term “halogen” is used to include fluorine, chlorine, bromine, and iodine.

Alternatively, R is selected from substituted or unsubstituted C₆-C₂₀ carbocyclic hydroxyaryl, substituted or unsubstituted hydroxy-4H-pyran-4-on-2-yl and substituted or unsubstituted C₄-C₂₀ hydroxyheteroaryl wherein the heteroatoms are selected from sulfur, nitrogen, and oxygen.

The aryl groups which R may represent may include phenyl, naphthyl, or anthracenyl and phenyl, naphthyl, or anthracenyl substituted with a hydroxyl group and one to three additional substituents selected from C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, C₂-C₆-alkoxycarbonyl, C₂-C₆-alkanoylamino and —O—R³, S—R³, —SO₂—R³, —NHSO₂R³ and —NHCO₂R³, wherein R³ is phenyl, naphthyl, or phenyl or naphthly substituted with one to three groups selected from C₁-C₆-alkyl, C₆-C₁₀ aryl, C₁-C₆-alkoxy, and halogen.

The heteroaryl groups which R may represent include a 5- or 6-membered hydroxy-substituted aromatic ring containing one to three heteroatoms selected from oxygen, sulfur, and nitrogen. Examples of such heteroaryl groups are hydroxythienyl, hydroxyfuryl, hydroxypyrrolyl, hydroxyimidazolyl, hydroxypyrazolyl, hydroxythiazolyl, hydroxyisothiazolyl, hydroxyoxazolyl, hydroxyisoxazolyl, hydroxytriazolyl, hydroxythiadiazolyl, hydroxyoxadiazolyl, hydroxytetrazolyl, hydroxypyridyl, hydroxypyrimidyl, hydroxybenzoxazolyl, hydroxybenzothiazolyl, hydroxy benzimidazolyl, hydroxyindolyl, and the like. The heteroaryl moiety may be substituted, for example, with up to three additional groups such as C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino. The heteroaryl moiety also may be substituted with a fused ring system, e.g., a benzo or naphtho residue, which may be unsubstituted or substituted, for example, with up to three of the groups set forth in the preceding sentence.

The hydroquinone derivatives are prepared by a reaction represented by the following:

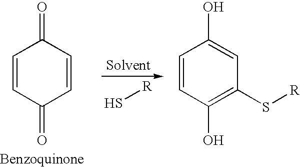

The process comprises reacting benzoquinone with a thiol containing R-group in the presence of an organic solvent (e.g. methanol, ethanol, isopropyl alcohol, methylene chloride, chloroform) or a mixed organic solvent. The reaction is typically allowed to run to completion, usually about 30 minutes. Shorter or longer reaction times may be required and can be determined by those of ordinary skill in the art. Upon completion of the reaction, the resulting derivative can be isolated and purified by techniques known to those of ordinary skill in the art (e.g. flash chromatography, etc.).

Typical compositions of the invention contain from about 4.00% to about 0.01% by weight, from about 2.00% to about 0.10% by weight, and even from about 1.00% to about 0.50% by weight, hydroquinone derivative. Lower concentrations may be employed for less pronounced hyperpigmentation conditions and in sunscreens and sunblocks used after skin brightening treatment, and higher concentrations may be employed with more acute pigmentation conditions. Suggested ranges also depend upon any adjunct ingredients employed in the compositions and the user's coloring and skin type as well as the extent of severity of the hyperpigmentation problem.

The skin brightening compositions of the invention may also contain other skin brightening ingredients in addition to hydroquinone derivative. These other ingredients include, but are not limited to, tetronic acid, tetronic acid derivatives, hydroquinone, kojic acid, 4-hydroxybenzyl alcohol, gallic acid, arbutin, α-hydroxyl acids, and fatty acid esters of ascorbic acid. Such other ingredients are known to those of skill in the art.

Typically, topical application to skin sites is accomplished in association with a carrier, and particularly one in which the active ingredient is soluble per se or is effectively solubilized (e.g., as an emulsion or microemulsion). Where employed, the carrier is inert in the sense of not bringing about a deactivation or oxidation of active or adjunct ingredient(s), and in the sense of not bringing about any adverse effect on the skin areas to which it is applied. For example, the compounds according to the present invention are applied in admixture with a dermatologically acceptable carrier or vehicle (e.g., as a lotion, cream, ointment, soap, stick, or the like) so as to facilitate topical application and, in some cases, provide additional beneficial effects as might be brought about, e.g., by moisturizing of the affected skin areas. While the carrier for dermatological compositions can consist of a relatively simple solvent or dispersant such as water, it is generally preferred that the carrier comprise a composition more conducive to topical application. In particular, a dermatological composition which will form a film or layer on the skin to which it is applied so as to localize the application and provide some resistance to washing off by immersion in water or by perspiration and/or aid in the percutaneous delivery of the active agent. Many preparations are known in the art, and include lotions containing oils and/or alcohols and emollients such as olive oil, hydrocarbon oils and waxes, silicone oils, other vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic, or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermally, dermatologically, or pharmaceutically acceptable carriers.

Suitable carriers include water, alcohols, oils and the like, chosen for their ability to dissolve or disperse ingredients used in the treatment. In some embodiments, active and/or adjunct ingredients are added to a sunscreen or sunblock formulations so that topical application has the further advantage of preventing repigmentation during and/or after treatment. Preferred formulae of this type are SPF 15 or higher. Many of these preferred embodiments contain titanium dioxide or zinc oxide which additionally soothe and lubricate the skin and help minimize side effects in sensitive skin and with formulations containing high concentrations of bleaching ingredients.

Generally in the practice of methods of the invention, the composition is topically applied to darker skin areas on a subject in a predetermined or as-needed regimen either at intervals by application of a lotion or the like, it generally being the case that gradual brightening is noted with each successive application. Insofar as has been determined based upon in vitro studies, no adverse side effects are encountered.

### EXAMPLES

While it is to be understood that modification of the synthetic method (i.e. order of addition, amounts of reagents, etc.) may change as a function of the particular derivative to be prepared, general synthetic methodology may be summarized as follows:

### Method A:

L-cysteine-hydrochloride mono-hydrate (5.95 g, 33.9 mmol)—or corresponding thiol starting material—was weighed out into a round bottom flask (500 mL) containing a stir bar and diluted (dissolved) with absolute ethanol (50 mL) and water (75 mL). The reaction contents were stirred. After 3-to-5 min of stirring, the green benzoquinone (3.6 g) slurry, prepared by diluting with ethanol (25 mL), was then added in portions over 2-to-3 min. Almost instantaneously (<1 min), the colored reaction mixture (green) turned to a deep purple (for some derivatives, it is orange/red). After 30 min of stirring (for some derivatives stirring is >30 min), the reaction mixture was concentrated under reduced pressure to afford crude product which was subsequently purified via column chromatography.

### Method B:

Similar to ‘Method A’, benzoquinone (3.6 g) was weighed out into a round bottom flask (500 mL) containing a stir bar and diluted with ethanol (50 mL). To a separate round bottom, cysteine-hydrochloride mono-hydrate (5.95 g, 33.9 mmol)—or corresponding thiol starting material—was weighed out and diluted (dissolved) with absolute ethanol (50 mL) and water (75 mL). Thereafter, to the green benzoquinone slurry, the thiol solution was added over 1-to-2 min. As observed using ‘Method A’, almost instantaneously (<1 min), the colored reaction mixture (green) turned to a deep purple (for some derivatives, it is orange/red). After 30 min of stirring (for some derivatives stirring is >30 min), the reaction mixture was concentrated under reduced pressure to afford crude product which was subsequently purified via column chromatography.

General in vitro tyrosinase inhibition assay:

EC₅₀ is the drug concentration that provokes an inhibition response half-way between the baseline and maximum. The EC₅₀ for the potential ‘Tyrosinase Inhibitors’ were conducted following known procedures (for example, Curto et al, 1999) as follows:
i) First, the compound of interest was evaluated for solubility in an aqueous environment.
ii) A concentrated stock solution was prepared in either water or dimethyl sulfoxide (DMSO).
iii) A wide range of dilutions were prepared from the stock solution; usually to measure the final inhibitor concentrations in a range of 10 nM to 10 mM.
iv) Assays were performed using Beckman Coulter DU 800 UV/V is Spectrophotometer.

### Enzyme Assay Conditions:

i) The tyrosinase activity was monitored by measuring the oxidation of 3,4-dihydroxy-L-phenylalanine (L-Dopa) to dopachrome at 475 nm, 30° C.

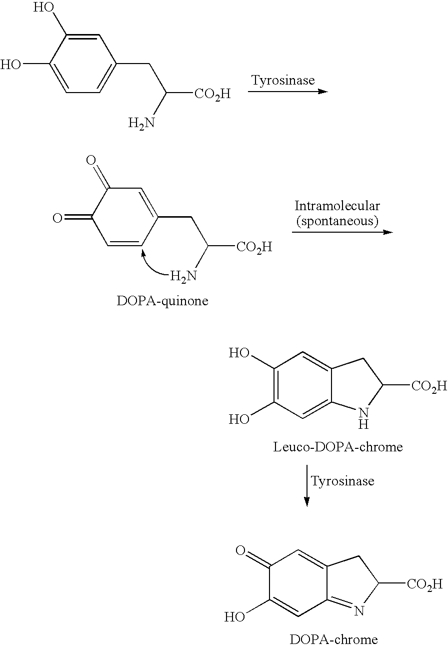

ii) The assay system was 1000 uL containing 50 mM Na₂HPO₄/NaH₂PO₄ at pH 7.0, 0.5 mM L-Dopa, and 18 Units of Mushroom Tyrosinase (Sigma T3824).
iii) Baseline initial rate for the tyrosinase activity was measured, then aliquot of inhibitor added and the change in slope/rate recorded.
iv) To minimize a final volume change, inhibitor was delivered as a 25 uL aliquot.
v) Inhibitory effects of DMSO solvent minimized by limiting final concentration to 2.5% (where relevant)—for each assay, background inhibition was accounted for with a DMSO blank.
vi) Microsoft Excel and Prism 4.02™ (GraphPad Software, Inc.; San Diego, Calif.) were used to calculate EC₅₀ values.
   Reference: E. V. Curto, C. Kwong, H. Hermersdorfer, H. Glatt, C. Santis, V. Virandor, V. J. Hearing, Jr., T. P. Dooley (1999) Inhibitors of Mammalian Melanocytes Tyrosinase: In Vitro Comparisons of Alkyl Esters of Gentistic Acid with Other Putative Inhibitors. Biochemical Pharmacology 57: 663-672.

### Compounds:

The following compounds were prepared and represent various examples of hydroquinone derivatives. The following compound examples are in no way intended to represent the full scope of the present invention.

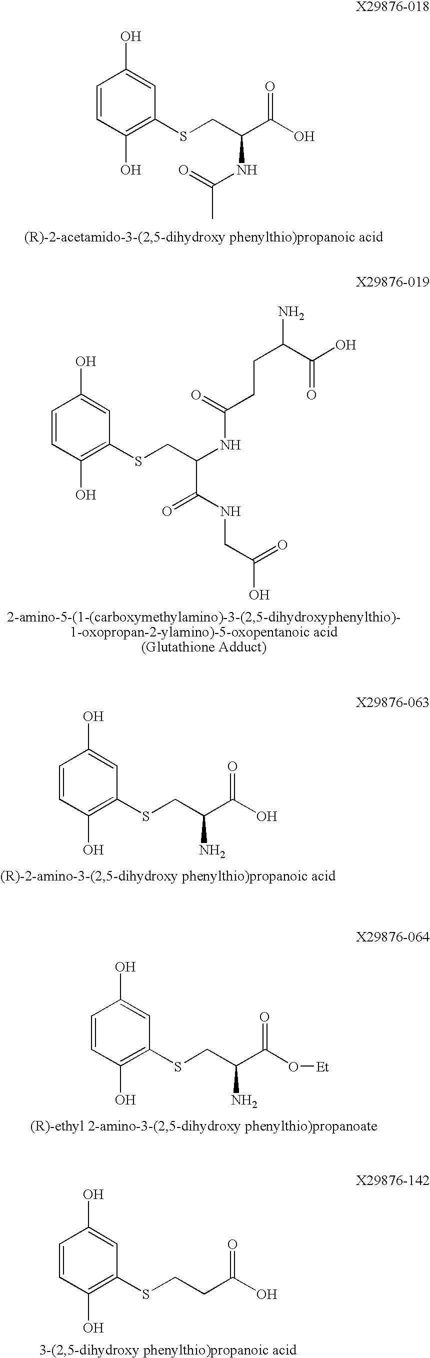

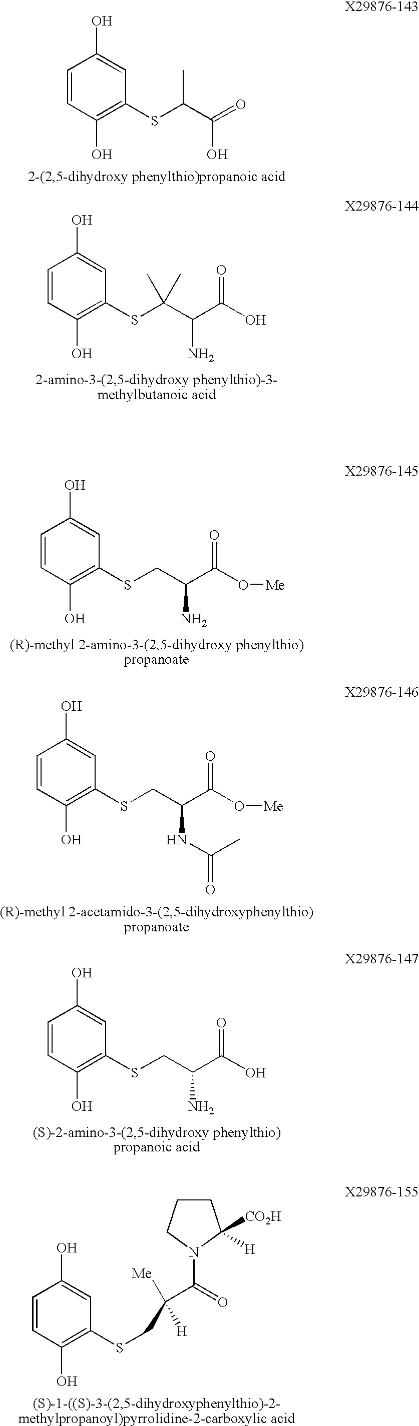

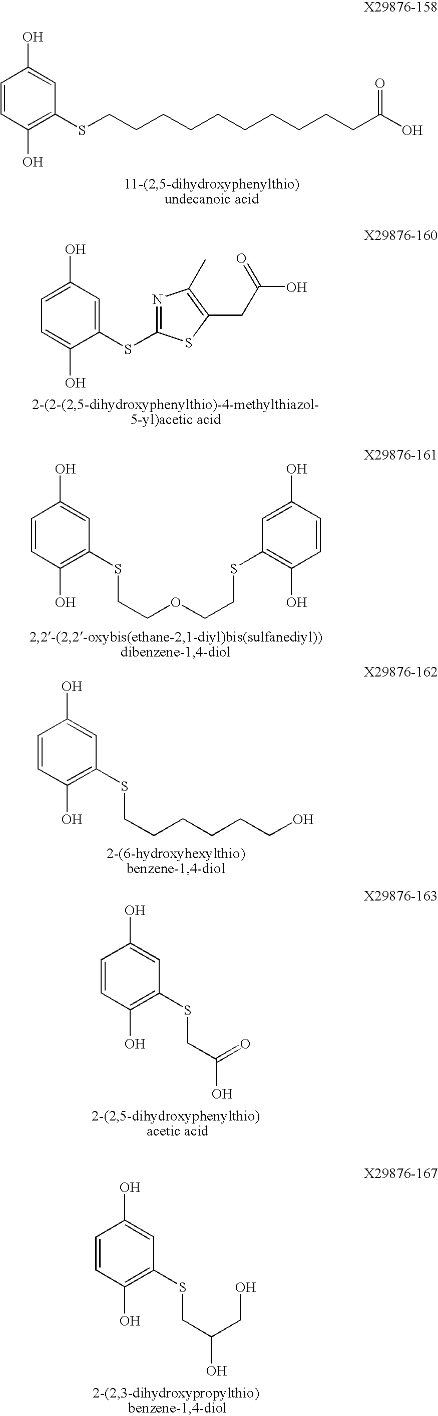

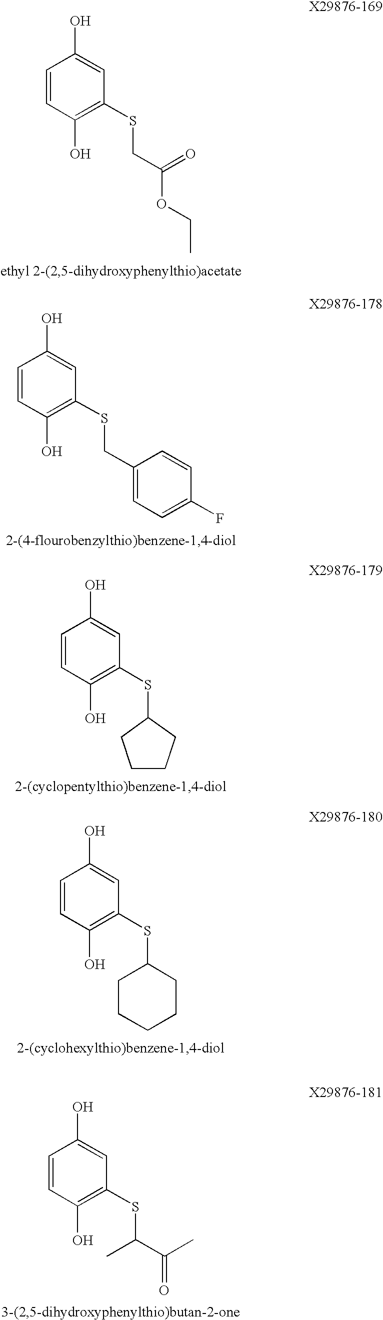

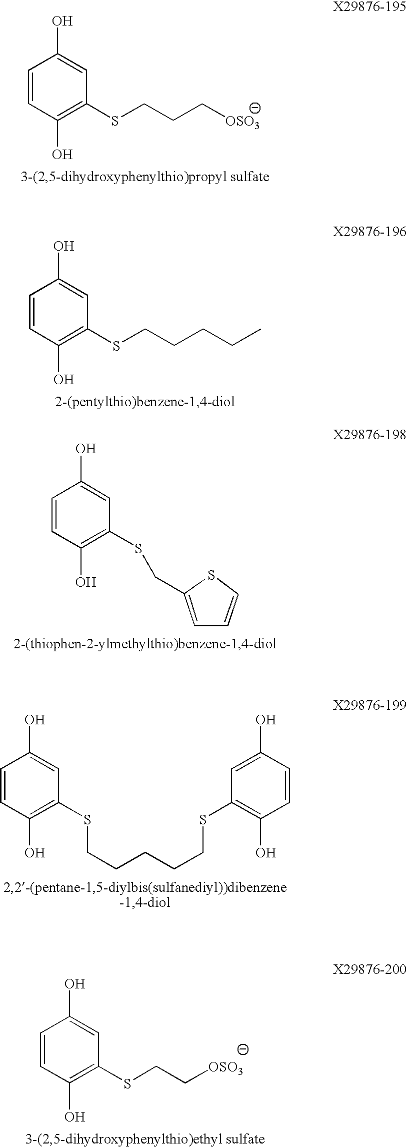

### EC₅₀ Data Summary:

The example below shows the in vitro tyrosine inhibition graph for hydroquinone.

The example below shows the in vitro tyrosine inhibition graph for X29876-063.

The table below summarizes a variety of the in vitro tyrosinase assay results:

| Compound | EC₅₀ mM |
|---|---|
| Hydroquinone | 0.62 |
| X29876-018 | 4.85 |
| X29876-019 | 10.40 |
| X29876-064 | 0.25 |
| X29876-063 | 0.02 |
| X29876-147 | 0.03 |
| X29876-155 | 12.20 |
| X29876-144 | 0.07 |
| X29876-145 | 0.13 |
| X29876-160 | 0.52 |
| X29876-162 | 0.01 |
| X29876-163 | 9.41 |
| X29876-169 | 3.28 |
| X29876-178 | 0.01 |
| X29876-181 | 0.31 |
| X29876-198 | 0.20 |
| X29876-200 | 4.81 |

The graph below is presented to help illustrate that, when compared to HQ, a variety of the hydroquinone derivatives have a more potent in vitro activity (lower EC₅₀ value). These experimental data help to elucidate structure-activity requirements. For example, in this in vitro assay, compound X29876-063 was about 25-fold more potent than HQ. If this were to equally equate to an in vivo formulation comparison, then the 2% HQ may be (theoretically) replaced by the novel material at only 0.08%.

The invention has been described in detail with particular reference to preferred embodiments thereof, but it will be understood that variations and modifications can be effected within the spirit and scope of the invention.

We claim:

1. A hydroquinone derivative represented by the following formula: 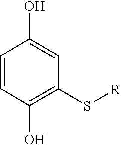
wherein R is a moiety selected from the group consisting of an alkyl, an alkenyl, a dienyl, a trienyl, and a tetraenyl containing up to about 20 carbon atoms, or
R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

2. The hydroquinone derivative according to claim 1, wherein said moiety is substituted with one to three groups selected from C₁-C₆-alkoxy, cyano, C₂-C₆-alkoxycarbonyl, C₂-C₆-alkanoyloxy, hydroxy, aryl, heteroaryl, thiol, thioether, dithiolane, and halogen

3. The hydroquinone derivative according to claim 1, wherein an aryl portion of R includes:
a) phenyl, naphthyl, or anthracenyl, and
b) phenyl, naphthyl, or anthracenyl substituted with a hydroxyl group and one to three additional substituents selected from the group consisting of C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, C₂-C₆-alkoxycarbonyl, C₂-C₆-alkanoylamino, —O—R³, S—R³, —SO₂—R³, —NHSO₂R³ and —NHCO₂R³, wherein R³ is phenyl, naphthyl, or phenyl or naphthly substituted with one to three groups selected from C₁-C₆-alkyl, C₆-C₁₀ aryl, C₁-C₆-alkoxy, and halogen.

4. The hydroquinone derivative according to claim 1, wherein a heteroaryl portion of R includes a 5- or 6-membered hydroxy-substituted aromatic ring containing one to three heteroatoms selected from the group consisting of oxygen, sulfur, and nitrogen.

5. The hydroquinone derivative according to claim 4, wherein said heteroaryl portion is selected from the group consisting of hydroxythienyl, hydroxyfuryl, hydroxypyrrolyl, hydroxyimidazolyl, hydroxypyrazolyl, hydroxythiazolyl, hydroxyisothiazolyl, hydroxyoxazolyl, hydroxyisoxazolyl, hydroxytriazolyl, hydroxythiadiazolyl, hydroxyoxadiazolyl, hydroxytetrazolyl, hydroxypyridyl, hydroxypyrimidyl, hydroxybenzoxazolyl, hydroxybenzothiazolyl, hydroxy benzimidazolyl, and hydroxyindolyl.

6. The hydroquinone derivative according to claim 4, wherein said heteroaryl portion is substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

7. The hydroquinone derivative according to claim 4, wherein said heteroaryl portion is substituted with a benzo residue or a naphtho residue optionally substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

8. A composition for brightening skin, comprising a hydroquinone derivative represented by the following formula: 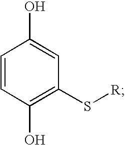
and
a cosmetically acceptable carrier,
wherein R is a moiety selected from the group consisting of an alkyl, an alkenyl, a dienyl, a trienyl, and a tetraenyl containing up to about 20 carbon atoms, or
R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

9. The composition according to claim 8, wherein said moiety is substituted with one to three groups selected from C₁-C₆-alkoxy, cyano, C₂-C₆-alkoxycarbonyl, C₂-C₆-alkanoyloxy, hydroxy, aryl, heteroaryl, thiol, thioether, dithiolane, and halogen.

10. The composition according to claim 8, wherein an aryl portion of R includes:
a) phenyl, naphthyl, or anthracenyl, and
b) phenyl, naphthyl, or anthracenyl substituted with a hydroxyl group and one to three additional substituents selected from the group consisting of C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, C₂-C₆-alkoxycarbonyl, C₂-C₆-alkanoylamino, —O—R³, S—R³, —SO₂—R³, —NHSO₂R³ and —NHCO₂R³, wherein R³ is phenyl, naphthyl, or phenyl or naphthly substituted with one to three groups selected from C₁-C₆-alkyl, C₆-C₁₀ aryl, C₁-C₆-alkoxy, and halogen.

11. The composition according to claim 8, wherein an heteroaryl portion of R includes a 5- or 6-membered hydroxy-substituted aromatic ring containing one to three heteroatoms selected from the group consisting of oxygen, sulfur, and nitrogen.

12. The composition according to claim 11, wherein said heteroaryl portion is selected from the group consisting of hydroxythienyl, hydroxyfuryl, hydroxypyrrolyl, hydroxyimidazolyl, hydroxypyrazolyl, hydroxythiazolyl, hydroxyisothiazolyl, hydroxyoxazolyl, hydroxyisoxazolyl, hydroxytriazolyl, hydroxythiadiazolyl, hydroxyoxadiazolyl, hydroxytetrazolyl, hydroxypyridyl, hydroxypyrimidyl, hydroxybenzoxazolyl, hydroxybenzothiazolyl, hydroxy benzimidazolyl, and hydroxyindolyl.

13. The composition according to claim 11, wherein said heteroaryl portion is substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

14. The composition according to claim 11, wherein said heteroaryl portion is substituted with a benzo residue or a naphtho residue optionally substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

15. The composition according to claim 8, wherein said hydroquinone derivative is present in an amount of from about 4.00% to about 0.01% by weight.

16. The composition according to claim 15, wherein said hydroquinone derivative is present in an amount of from about 2.00% to about 0.10% by weight.

17. The composition according to claim 16, wherein said hydroquinone derivative is present in an amount of from about 1.00% to about 0.50% by weight.

18. A method of brightening skin, comprising applying the composition according to claim 8 to skin.

19. The method according to claim 18, wherein said composition is applied to skin in a predetermined regimen or in a as-needed regimen until a desired level of skin brightening is achieved.

20. A method of making a hydroquinone derivative comprising reacting benzoquinone with a molecule having the structure SH—R in the presence of an organic solvent or a mixed organic solvent,
wherein R is a moiety selected from the group consisting of an alkyl, an alkenyl, a dienyl, a trienyl, and a tetraenyl containing up to about 20 carbon atoms, or
R is selected from the group consisting C₆-C₂₀ carbocyclic hydroxyaryl, hydroxy-4H-pyran-4-on-2-yl and C₄-C₂₀ hydroxyheteroaryl wherein the heteroatoms are at least one of sulfur, nitrogen, and oxygen.

21. The method according to claim 20, wherein said moiety is substituted with one to three groups selected from C₁-C₆-alkoxy, cyano, C₂-C₆-alkoxycarbonyl, C₂-C₆-alkanoyloxy, hydroxy, aryl, heteroaryl, thiol, thioether, dithiolane, and halogen.

22. The method according to claim 20, wherein an aryl portion of R includes:
a) phenyl, naphthyl, or anthracenyl, and
b) phenyl, naphthyl, or anthracenyl substituted with a hydroxyl group and one to three additional substituents selected from the group consisting of C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, C₂-C₆-alkoxycarbonyl, C₂-C₆-alkanoylamino, —O—R³, S—R³, —SO₂—R³, —NHSO₂R³ and —NHCO₂R³, wherein R³ is phenyl, naphthyl, or phenyl or naphthly substituted with one to three groups selected from C₁-C₆-alkyl, C₆-C₁₀ aryl, C₁-C₆-alkoxy, and halogen.

23. The method according to claim 20, wherein an heteroaryl portion of R includes a 5- or 6-membered hydroxy-substituted aromatic ring containing one to three heteroatoms selected from the group consisting of oxygen, sulfur, and nitrogen.

24. The method according to claim 23, wherein said heteroaryl portion is selected from the group consisting of hydroxythienyl, hydroxyfuryl, hydroxypyrrolyl, hydroxyimidazolyl, hydroxypyrazolyl, hydroxythiazolyl, hydroxyisothiazolyl, hydroxyoxazolyl, hydroxyisoxazolyl, hydroxytriazolyl, hydroxythiadiazolyl, hydroxyoxadiazolyl, hydroxytetrazolyl, hydroxypyridyl, hydroxypyrimidyl, hydroxybenzoxazolyl, hydroxybenzothiazolyl, hydroxy benzimidazolyl, and hydroxyindolyl.

25. The method according to claim 23, wherein said heteroaryl portion is substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

26. The method according to claim 23, wherein said heteroaryl portion is substituted with a benzo residue or a naphtho residue optionally substituted with up to three groups selected from the group consisting of C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino.

27. The method according to claim 20, further comprising isolating and purifying at least one resulting derivative.
